(19) United States
(12) Patent Application Publication
(10) Pub. No.: US 2020/0237550 A1
(43) Pub. Date: Jul. 30, 2020

(54) OSTOMY SKIN BARRIER ADAPTER

(71) Applicant: Maine Medical Center, Portland, ME (US)

(72) Inventors: Robert K. Hussey, Waterville, ME (US); Daniel Hussey, Windham, ME (US)

(21) Appl. No.: 16/744,256

(22) Filed: Jan. 16, 2020

Related U.S. Application Data
(60) Provisional application No. 62/793,023, filed on Jan. 16, 2019.

(51) Int. Cl. A61F 5/449

(52) U.S. Cl. CPC A61F 5/449

(57) ABSTRACT

An adapter for securing a skin barrier for ostomy collection allows placement of the skin barrier in a variety of positions to avoid interfering with surgical incisions, bandages and/or drains. An oblong or elliptical shaped skin barrier may be rotated or oriented to an alternate position and attached to a belt using the adapter. The adapter has a linkage for receiving the belt, typically a set of pins or protrusions, and another linkage for engaging the skin barrier through the same apertures intended to receive the belt at an alternate position. The adapter allows the oblong skin barrier to be rotated to a position that avoids incisions or surgical structures without cutting an adhesive region and compromising adhesion of the skin barrier. The adapter provides full retention capability of the adhesive and the support belt are for maintaining a fluidic barrier to the ostomy collection bag or pouch.

### RELATED APPLICATIONS

This patent application claims the benefit under 35 U.S.C. § 119(e) of U.S. Provisional Patent App. No. 62/793,023, filed Jan. 16, 2019, entitled “OSTOMY SKIN BARRIER ADAPTER,” incorporated herein by reference in entirety.

### BACKGROUND

A stoma is a surgically created exit from the human gastrointestinal (GI) tract resulting from failure or treatment of a portion of the GI tract. Different procedures are employed, depending on whether the treated ailment affects the small intestine, large intestine, or bowel, for example, however result in a similar need to collect gastric contents from an interrupted GI tract.

Ostomy collection systems are post-surgical arrangements for collection of these gastric contents responsive to the surgically diverted GI stream. An ostomy pouching system is a prosthetic medical device that externally interfaces with the stoma, or other surgically created exit, for retaining expelled material into a pouch or bag from which they may be disposed. Pouching systems are often associated with colostomies, ileostomies, and urostomies, similar procedures such as gastrostomies, cholecystostomies, and jejunostomies may result in a stoma. A typical process employs a skin barrier that interfaces with a dermal surface of a patient by adhesive and belted means. The skin barrier is disposed at the stoma exit for receiving expelled material and provides a receptacle for attachment to the collection bag.

### SUMMARY

An adapter for securing a skin barrier for ostomy collection allows placement of the skin barrier in a variety of positions to avoid interfering with surgical incisions, bandages and/or drains. A support belt is often employed with the skin barrier for supplementing the skin adhesive on the barrier, however the conventional skin barrier must be oriented in a particular manner to engage the belt. An oblong or elliptical shaped skin barrier may be rotated or oriented to an alternate position and attached to the belt using an adapter. The adapter has a linkage for receiving the belt, typically a set of pins or protrusions, and another linkage for engaging the skin barrier through the same apertures intended to receive the belt, but at an alternate position. An adapter disposed between the belt and skin barrier allows the oblong skin barrier to be rotated to a position that avoids incisions or surgical structures without cutting an adhesive region of the skin barrier, which would otherwise compromise adhesion of the skin barrier. In this manner, full retention capability of the adhesive and the support belt are provided for maintaining a fluidic barrier to the ostomy collection bag or pouch.

Configurations herein are based, in part, on the observation that standard ostomy collection appliance employ adhesives and belts that impose a particular orientation on the patient/wearer. Unfortunately, conventional approaches to skin barrier adapters suffer from the shortcoming that the predetermined positioning of the oblong skin barrier relative to a supportive belt may interfere with other surgical artifacts, such as incisions, bandages and drains. Conventional skin barriers have an adhesive region, which is often cut in order to accommodate space around an incision, drain or bandage. For example, patients with an ostomy who undergo a laparotomy and use a typical conventional skin barrier are encumbered because the barrier's interface with the belt is longitudinally oriented and oval-shaped. Accordingly, configurations herein substantially overcome the shortcomings of conventional ostomy collection by providing an adapter between the belt and the skin barrier to provide rotational communication with the belt attachment to allow alternate rotational positioning of the oblong skin barrier for avoiding interference with other medical or surgical conditions while retaining a secure fluidic engagement for transporting expelled biological waste to the ostomy bag. An elliptical, oblong or elongated skin barrier may therefore be accommodated without cutting by simply rotating or repositioning to a location that does not interfere with the surgical sites.

Configurations depicted below illustrate an adapter for securing a post-surgical assistance appliance for a surgical stoma, including a linkage to a support belt, and a linkage to a skin barrier. The skin barrier has a receptacle for engaging an ostomy collection repository in a sealing engagement with a surgical stoma to receive gastric contents. The linkage to the support belt has a fixed orientation relative to the support belt, however the linkage to the skin barrier has a variable orientation based on an arrangement of protrusions and receptacles securing the skin barrier in a predetermined orientation with respect to the support belt. The linkage to the skin barrier adapted to engage the skin barrier in a plurality of position relative to the support belt.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIGS. 1A and 1B show a prior art configuration of a conventional skin barrier for use with an ostomy collection bag;

FIGS. 2A-2D show an ostomy belt adapter as disclosed herein for orienting the skin adapter at a plurality of orientation;

FIGS. 3A-3C show an alternate configuration with a unitary flange for surrounding the receptacle; and

FIGS. 4A-4D show a further configuration adapted for installing the skin barrier at angular increments relative to the belt.

### DETAILED DESCRIPTION

Depicted below are several examples of the adapter for repositioning and/or rotating the skin barrier away from surgical sites while still engaging an abdominal support belt for securing the ostomy collection appliance (bag or pouch). Other configurations for utilizing the disclosed approach for securing the ostomy bag to the relocated skin barrier may be apparent.

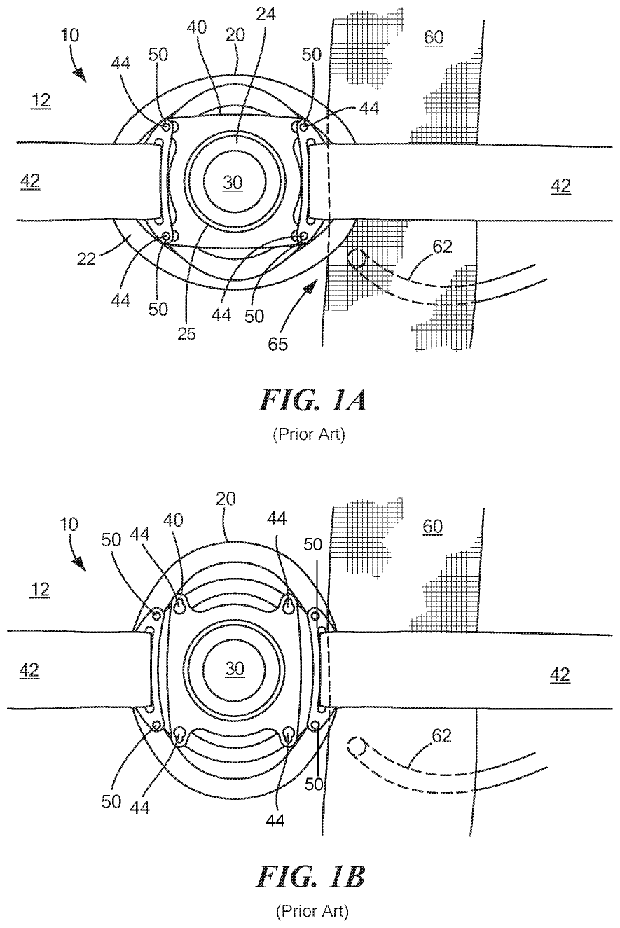

FIG. 1 shows a prior art configuration of a conventional skin barrier for use with an ostomy collection bag. Referring to FIG. 1, a conventional ostomy collection system 10 includes a skin barrier 20 for engaging a surgical stoma 30 on a dermal surface of a patient abdomen 12. The skin barrier 20 includes an adhesive region 22 for akin adhesion and a receptacle 24 for engaging the stoma 30 for transfer of gastric contents. The receptacle 24 includes a ridge 25 or recession for engagement with a collection bag or pouch. The receptacle 24 is also surrounded by a belt retainer 40 for attachment to a belt 42 to provide additional support to the skin barrier 20. The belt retainer 40 has apertures 44 for receiving pins 50 secured to a loop at an end of the belt 42.

The adhesive region 22 of the conventional skin barrier 20 has an oblong or elliptical shape that can interfere with a bandage 60 protecting a surgical incision and a surgical drain 62 which is often required post surgery, defined by an interference region 65. A conventional design only allows one orientation with a belt. To avoid the bandage the barrier would have to be trimmed which reduces adhesive contact area. The pins 50 have a fixed arrangement which can only retain the apertures 44 in a fixed position which prevents rotation of the skin barrier, as shown in FIG. 1B. The rotated belt retainer 40′ exhibits a wider pitch 48 between the receptacles 44 which do not align with the conventional pins 50 attached to the belt 42.

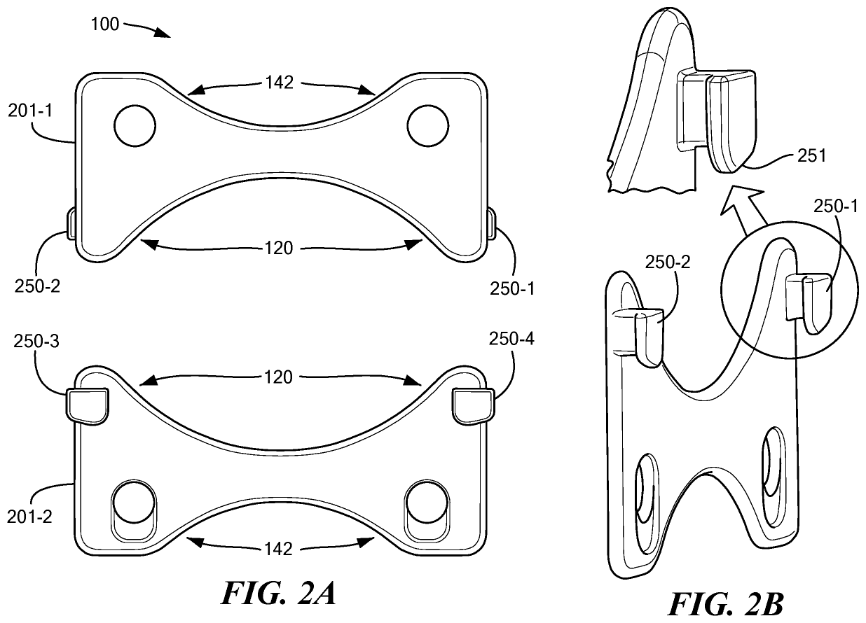

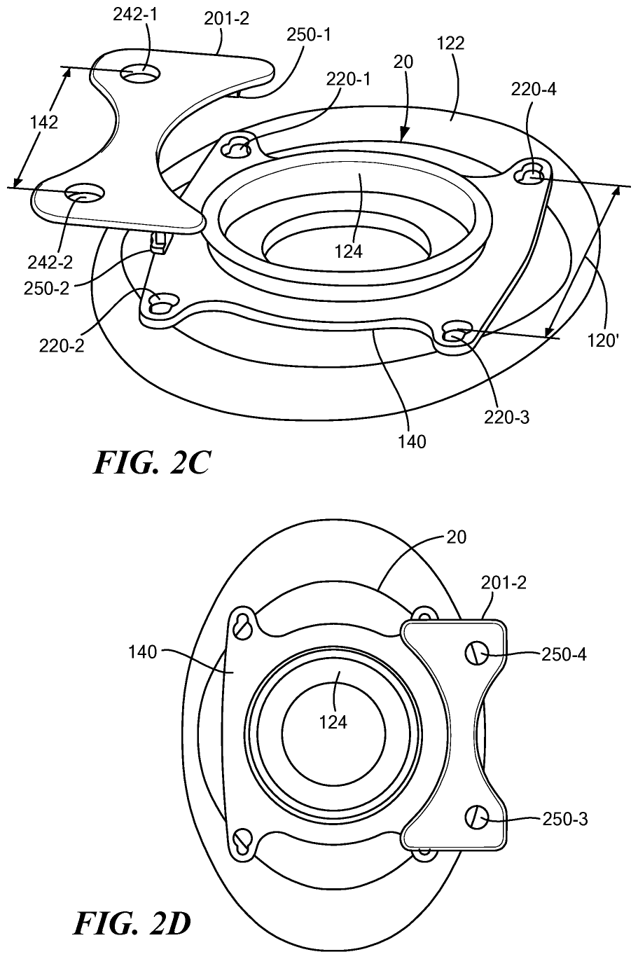

FIGS. 2A-2D show an ostomy belt adapter 100 as disclosed herein for orienting the skin barrier 20 at a plurality of orientation. Referring to FIGS. 2A-2D, the adapter 100 secures a post-surgical assistance appliance for a surgical stoma, such as a collection nag or pouch. The adapter 100 is disposed between the support belt 42 and the skin barrier 20, and includes a linkage 142 to the support belt 42 and a linkage 120 to the skin barrier 20. The linkage 120 to the skin barrier is adapted to engage the skin barrier 20 in one or more alternate positions relative to the support belt 42 so that the skin barrier 20 may be positioned to avoid surgical incisions, bandages and drains. The skin barrier 20 includes a receptacle 124 for engaging an ostomy collection repository in a sealing engagement with a stoma 30, discussed further below.

FIG. 2C shows the linkage 142 to the support belt defined by a plurality of apertures 242-1 . . . 242-2 (242 generally). A pitch distance 120′ is based on an arrangement of apertures 220-1 . . . 220-2 (220 generally) on the skin barrier 20, and the linkage 120 to the skin barrier defines a plurality of protrusions 250-1 . . . 250-4 based on an arrangement of protrusions on the support belt 42, defined by distance 142′. In other words, the skin barrier 20 has a horizontal spacing, or pitch, between the apertures, 220, and a different vertical pitch (relative to a standing patient). The conventional support belt 42 employs protrusions 50 spaced to match the horizontally oriented skin barrier, and a 90 degree rotation to the vertical orientation results in a different spacing. The adapter 100 receives the protrusions 50 from the belt 42 and attaches to the greater pitch distance 120′ of the apertures 220 on the rotated skin barrier 20 by employing protrusions 250-1 . . . 250-4 (250 generally).

Continuing to refer to FIG. 2C, the skin barrier 20 includes a supporting flange 140, an ostomy receptacle 124, and an adhesive region 122, such that the ostomy receptacle 124 is adapted for engagement to the ostomy collection repository (bag or pouch) for receiving gastric contents. The supporting flange 140 is disposed for retaining the ostomy receptacle 124 in communication with the stoma 30, such that the linkage 120 to the skin barrier 20 is defined by the supporting flange 140 and secured by the support belt 42 or other reinforcement.

Referring to FIGS. 1A-2D, as can be seen in the configuration of FIGS. 2A-2D, the adapter 100 includes opposed planar elements 201-1 . . . 201-2 (201, generally) flanking the supporting flange 140 for engaging the respective apertures 220 on the supporting flange. The linkage 142 to the support belt therefore includes one or more protrusions or pins 50, and a corresponding number of receptacles 242 for receiving a respective pin 50. The linkage 120 to the skin barrier further includes one or more protrusions 250 and a corresponding number of receptacles or apertures 220 for receiving a respective protrusion. The protrusions 250 may also include a hook, tab or cleat 251 extending in the direction of belt tension for retaining the cleat 251 and protrusion 250 in the receptacle.

The installed adapter assembly therefore includes opposed planar elements 201, each having a plurality of mating receptacles (apertures 242) defining the linkage 142 to the support belt 42, such that the support belt 42 has a respective belt protrusion (pin 50) for each of the mating receptacles 242. Each of the opposed planar elements 201 also has a plurality of flange protrusions 250 (lugs, cleats, hooks) defining the linkage 120 to the supporting flange 140 of the skin barrier 20. Each flange protrusion 250 is adapted to engage a respective mating receptacle 220 on the supporting flange 140, typically two opposed pairs on either side of the supporting flange 140. In other words, the adapter 100 receives the pins 50 of the support belt 42 and transfers the load to a wider arrangement of protrusions 250 that engage the reoriented skin barrier through receptacles 220 positioned to avoid incisions, bandages and drains.

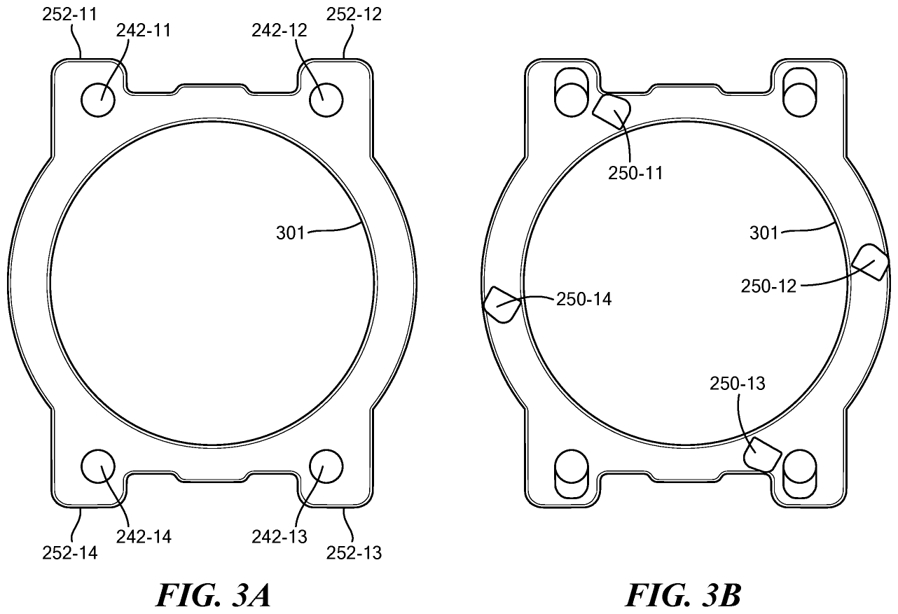

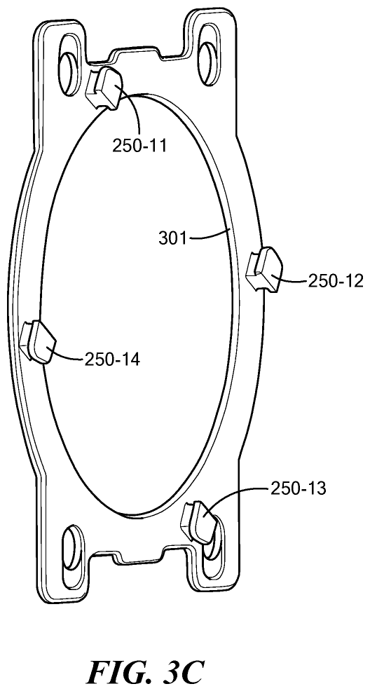

In another configuration, the adapter 100 takes the form of a unitary ring concentric or overlaying with the supporting flange 140. FIGS. 3A-3C show the alternate configuration with a unitary flange for surrounding the receptacle. Referring to FIGS. 1 and 3A-3C, FIG. 3A shows a top view of the concentric ring 301 for circumferentially engaging the supporting flange 140 to define the linkage to the skin barrier 20. The concentric ring 301 is disposed around the receptacle 124 for retention of the skin barrier 20 against a patient dermal surface, and defines a pattern of protrusions 250 for engaging corresponding receptacles 220 on the skin barrier. The linkage to the skin barrier therefore includes a plurality of flange protrusions 250-11 . . . 250-14 (250 generally) on the concentric ring 301, such that each flange protrusion 250 of the plurality of flange protrusions is adapted to engage a respective mating receptacle (apertures 220) on the supporting flange 140. The protrusions 250 define an angular layout corresponding to the apertures 220, such that the supporting flange 140, and correspondingly the skin barrier 20, are disposed at an orientation defined by the angular layout with respect to the belt 42.

The linkage to the support belt further comprising a pattern of tabs 252-11 . . . 252-14, each having an aperture responsive to engagement by the support belt 42, extending from the concentric ring 301. Each tab 252 has a respective mating receptacle 242 for receiving the belt pins 50, and are spaced accordingly, such that each of the mating receptacles 242 is adapted to engage a pin 50 in communication with the support belt 42.

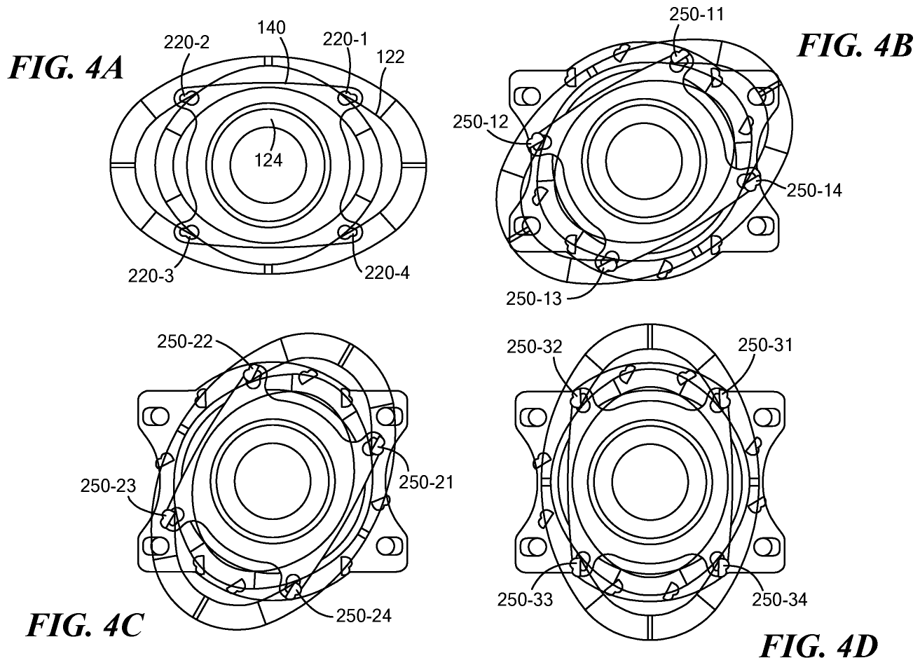

FIGS. 4A-4D show a further configuration adapted for installing the skin barrier at angular increments relative to the belt. Referring to FIGS. 3A-4D, the linkage 120 to the skin barrier includes an arrangement of variable positioning protrusions, such that the variable positioning protrusions are adapted to engage apertures on the skin barrier in multiple positions. FIGS. 4A-4D illustrate a progression such that the linkage to the skin barrier secures the skin barrier 20 in a plurality of rotational increments relative to the linkage 142 to the support belt 42. Multiple sets of protrusions 250 occupy positions on the concentric ring 201 to define different degrees of rotations. Any suitable granularity may be employed as long as sufficient distance between the protrusions 250 is provided for engaging the supporting flange 140 in a noninterfering manner. FIG. 4A shows the skin barrier 20 oriented long-side horizontal as would be for a conventional belt engagement. FIG. 4B shows the skin barrier positioned for receiving a first set of protrusions 250-11 . . . 250-14 for a rotation around 30 degrees. FIG. 4C shows a second set of protrusions 250-21 . . . 250-24 engaging the skin barrier for a 60 degree rotation, and FIG. 4D shows a third set of protrusions 250-31 . . . 250-34 for a 90 degree shift. Alternate arrangements of protrusions 250 provide that the pattern of protrusions each define a rotational configuration relative to the support belt 42, when the support belt is adapted to extend in a substantially horizontal plane around a patient abdominal region as it is typically worn. Alternative protrusion arrangements may be envisioned for various rotations, and alternate linkage mechanisms may be envisioned to secure the skin barrier at various degrees of rotation relative to the generally fixed horizontal belt orientation.

A number of protrusion and receptacle attachments are depicted above, including pins, protrusions, cleats and lugs, which engage apertures and mating receptacles. Reversal of the protrusion and corresponding receptacle may generally be performed to result in similar performance, and should not be deemed to deviate from the claimed approach herein.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. An adapter device for securing a post-surgical assistance appliance for a surgical stoma, comprising:
a linkage to a support belt; and
a linkage to a skin barrier, the skin barrier having a receptacle for engaging an ostomy collection repository in a sealing engagement with a stoma,
the linkage to the skin barrier adapted to engage the skin barrier in a plurality of position relative to the support belt.

2. The device of claim 1 wherein the linkage to the skin barrier includes a plurality of variable positioning protrusions, the variable positioning protrusions arranged in patterns adapted to engage apertures on the skin barrier in multiple rotations.

3. The device of claim 2 wherein the linkage to the support belt defines a plurality of apertures based on an arrangement of apertures on the skin barrier, and the linkage to the skin barrier defines a plurality of protrusions based on an arrangement of protrusions on the support belt.

4. The device of claim 1 wherein the skin barrier includes a supporting flange, an ostomy receptacle, and an adhesive region, the ostomy receptacle adapted for engagement to the ostomy collection repository and the supporting flange disposed for retaining the ostomy receptacle in communication with the stoma, the linkage to the skin barrier defined by the supporting flange.

5. The device of claim 1 wherein the linkage to the skin barrier secures the skin barrier in one or more alternate rotational increments relative to the linkage to the support belt.

6. The device of claim 1 further comprising a plurality of planar elements, the linkage to the support belt further comprising:
one or more protrusions and a corresponding number of receptacles for receiving a respective protrusion; and
the linkage to the skin barrier further comprising one or more protrusions and a corresponding number of receptacles for receiving a respective protrusion.

7. The device of claim 4 further comprising opposed planar elements, each of the opposed planar elements having a plurality of mating receptacles defining the linkage to the support belt, the support belt having a respective belt protrusion for each of the mating receptacles; and
each of the opposed planar elements having a plurality of flange protrusions defining a linkage to the supporting flange, each flange protrusion of the plurality of the flange protrusions adapted to engage a respective mating receptacle on the supporting flange.

8. The device of claim 4 further comprising:
a concentric ring, the concentric ring for circumferentially engaging the supporting flange to define the linkage to the skin barrier;
the linkage to the skin barrier further comprising a plurality of flange protrusions on the concentric ring, each flange protrusion of the plurality of flange protrusions adapted to engage a respective mating receptacle on the supporting flange; and
the linkage to the support belt further comprising a plurality of tabs extending from the concentric ring,
each tab of the plurality of tabs having a respective mating receptacle, each of the mating receptacles adapted to engage a protrusion in communication with the support belt.

9. The device of claim 1, further comprising:
a concentric ring, the concentric ring disposed around the receptacle for retention of the skin barrier against a patient dermal surface;
a pattern of protrusions for engaging corresponding receptacles on the skin barrier; and
a pattern of tabs having apertures responsive to engagement by the support belt.

10. The device of claim 9 wherein the pattern of protrusions defines a rotational configuration relative to the support belt, and
the support belt is adapted to extend in a substantially horizontal plane around a patient abdominal region.

11. The device of claim 9 further comprising a plurality of patterns of protrusions, each pattern of the plurality of protrusions adapted to engage the supporting flange at a predetermined angle of orientation.

12. An adapter for securing a post-surgical assistance appliance for a surgical stoma, comprising:
a linkage to a support belt; and
a linkage to a skin barrier, the skin barrier having a receptacle for engaging an ostomy collection repository in a sealing engagement with a stoma,
the linkage to the support belt having a fixed orientation relative to the support belt; and
the linkage to the skin barrier having a variable orientation based on an arrangement of protrusions and receptacles securing the skin barrier in a predetermined orientation with respect to the support belt, the linkage to the skin barrier adapted to engage the skin barrier in a plurality of position relative to the support belt.

13. In a surgical environment employing an oblong shaped skin barrier for interfacing an ostomy collection receptacle to a surgical stoma, the skin barrier having a predetermined fixed orientation to a belt for dermal surface positioning, an ostomy collector adapter device, comprising:
a plurality of belt receptacles for engaging an ostomy belt; and
a plurality of barrier receptacles for engaging an ostomy collector,
the barrier receptacles adapted to engage the ostomy collector at a plurality of positions relative to the belt receptacles.

14. The device of claim 13 wherein the adapter includes apertures based on a pitch of the skin barrier, the pitch defining a predetermined orientation of the skin barrier for attachment to the belt.

15. The device of claim 14 wherein the adapter accommodates a different pitch than the predetermined orientation.
